(19) United States
(12) Patent Application Publication
(10) Pub. No.: US 2025/0000986 A1
(43) Pub. Date: Jan. 2, 2025

(54) IMPROVED SOPHOROLIPID DERIVATIVES WITH THERAPEUTIC AGENT CARGOS AND THEIR USE

(71) Applicant: LOCUS SOLUTIONS IPCO, LLC, SOLON, OH (US)

(72) Inventors: SEAN FARMER, FT. LAUDERDALE, FL (US); LEE SPEIGHT, SOLON, OH (US); DANIEL HAGAMAN, MECHANICSVILLE, VA (US); ANDREW MORRIS, RICHMOND, VA (US); NICHOLAS CALLOW, SOLON, OH (US)

(21) Appl. No.: 18/249,245

(22) PCT Filed: Feb. 1, 2023
(86) PCT No.: PCT/US2023/012091

§ 371 Date: Apr. 15, 2023

Related U.S. Application Data
(60) Provisional application No. 63/305,804, filed on Feb. 2, 2022.

(51) Int. Cl. A61K 47/54

(52) U.S. Cl. CPC A61K 47/549

(57) ABSTRACT

The present invention provides modified sophorolipids of formula (I) incorporating therapeutic molecules as therapeutic agent cargos to improve the bioavailability and/or delivery of such agents. The modified sophorolipids of the present invention are capable of efficiently delivering the therapeutic agent cargos to the target site and enhancing the efficacy of the therapeutic agent cargos and/or the sophorolipids. Additionally, the present invention provides methods of treating skin conditions or ailments as well as of maintaining or restoring gut health in a subject.

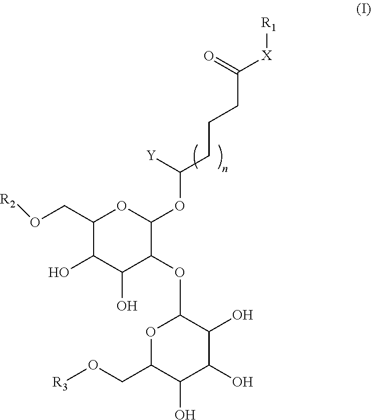

### CROSS-REFERENCE TO A RELATED APPLICATION

This application claims priority to U.S. provisional patent application No. 63/305,804 filed Feb. 2, 2022, the entire contents of which are incorporated herein by reference.

### BACKGROUND OF THE INVENTION

Sophorolipids are classified as glycolipid biosurfactants as they are produced by fermentation of microbes, such as Starmerella bombicola, using carbohydrate and lipids as carbon sources. Sophorolipids are amphiphilic molecules containing a hydrophilic carbohydrate group (sophorose) and a hydrophobic fatty acid. Sophorose is a disaccharide that consists of two glucose molecules linked via a β-1,2 bond. The sophorose in sophorolipids can be acetylated at the 6′—and/or 6″-positions. The fatty acid in sophorolipids is glycosidically attached to the sophorose molecule through its hydroxyl group, and its terminal carboxylic acid group is either free (as shown in FIG. 1) or internally esterified (i.e., lactonic form), generally at the 4″-position. The fatty acid of sophorolipids generally has 16 or 18 carbon atoms with one unsaturation. However, it can consist of chain lengths from C12 to C22 having various degrees and positions of unsaturation. Thus, sophorolipids are frequently produced as a mixture of related molecules. Differences among the related molecules arise mainly from: their fatty acid structure (degree of unsaturation, chain length, position(s) of unsaturation and position of hydroxylation); whether they are produced in the free or lactonic form, the acetylation pattern; the presence of stereoisomers; and/or whether the glycosidic bond on the fatty acid is at the @-position (e.g., terminal) or @-1 position (sub-terminal).

Sophorolipids have many advantageous characteristics that make them superior to synthetic surfactants, such as biodegradability, low toxicity, high surface and interfacial activities, and stability in under wide ranges of temperatures, pressures, and ionic strengths.

In recent years, chemical modifications of sophorolipids have been pursued with an eye toward enhancing the properties of sophorolipids. An example is poly(sophorolipid) which is a polymer of sophorolipids having potential biomaterials applications.

### BRIEF SUMMARY OF THE INVENTION

One aspect of the present invention provides sophorolipids modified with small molecule therapeutic agents having one or more hydroxyl or carboxyl functional groups to improve the bioavailability and/or delivery of such agents.

According to a first set of embodiments, the present invention provides modified sophorolipids of the formula (I) or salts thereof:

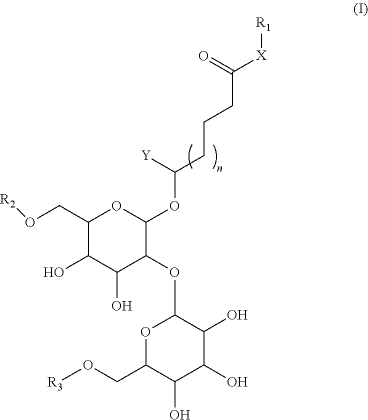

wherein
Y is hydrogen or methyl;
R₁ is a first therapeutic agent cargo linked to the sophorolipid structure via an ester linkage with X═O or an amide linkage with X═NR₀, wherein R₀ is hydrogen or a substituent;
R₂ and R₃ are each independently hydrogen or acetyl; and
n is an integer between 4 and 16 inclusively, preferably 12 when Y is methyl, and the chain defined by n has 0-3 unsaturation.

In some embodiments, R₁ is a first therapeutic agent cargo that forms an ester linkage.

In other embodiments, R₁ is a first therapeutic agent cargo that forms an amide linkage with X═NR₀, wherein R₀ is hydrogen, lower alkyl, aryl, or a combination thereof.

According to a second set of embodiments, the present invention provides modified sophorolipids of the formula (I) or salts thereof wherein:
Y is hydrogen or methyl;
R₁ is a first therapeutic agent cargo linked to the sophorolipid structure via an ester linkage with X═O or an amide linkage with X═NR₀, wherein R₀ is hydrogen or a substituent;
R₂ and R₃ are each independently hydrogen, acetyl, or a second therapeutic agent cargo linked to the sophorolipid structure via an ester linkage, provided that at least one of R₂ and R₃ is the second therapeutic agent; and
n is an integer between 4 and 16 inclusively, preferably 12 when Y is methyl, and the chain defined by n has 0-3 unsaturation,
wherein the first and second therapeutic agents are the same or different compounds.

In some embodiments, R₁ is a first therapeutic agent cargo that forms an ester linkage.

In other embodiments, R₁ is a first therapeutic agent cargo that forms an amide linkage with X═NR₀, wherein R₀ is hydrogen, lower alkyl, aryl, or a combination thereof.

According to a third set of embodiments, the present invention provides modified sophorolipids of the formula (1) or salts thereof wherein:
Y is hydrogen or methyl;
R₁ is hydrogen and X is O;
R₂ and R₅ are each independently hydrogen, acetyl, or a second therapeutic agent cargo linked to the sophorolipid structure via an ester linkage, provided that at least one of R₂ and R₃ is the second therapeutic agent cargo; and
n is an integer between 4 and 16 inclusively, preferably 12 when Y is methyl, and the chain defined by n has 0-3 unsaturation.

The present invention encompasses all of the compounds represented by the general formula (I), including salts thereof, hydrates thereof, geometric and optical isomers thereof, and polymorphic forms thereof.

Other aspects of the present invention are directed to a method of improving the bioavailability and dermal penetrability of the therapeutic agent cargos. The modified sophorolipids of the present invention are capable of efficiently delivering the therapeutic agent cargos to the target site and enhancing the efficacy of the therapeutic agent and/or the sophorolipids.

Additionally, certain embodiments of the present invention provide a method of treating skin conditions or ailments with a composition comprising the modified sophorolipid. Other embodiments of the present invention include a method of maintaining or restoring gut health, reducing pathogenic gram-negative bacteria, and/or improving weight gain.

### BRIEF DESCRIPTION OF THE DRAWINGS

FIG. 1 illustrates an exemplary general structure of free acidic forms of sophorolipids, wherein: Y is hydrogen or methyl; R and R′ are each independently hydrogen or acetyl; “Chain” denotes a saturated or unsaturated aliphatic hydrocarbon chain of typically about C10 to C20 in length, having various degrees and positions of unsaturation.

### DETAILED DESCRIPTION OF THE INVENTION

### Modified Sophorolipids

In one aspect, the present invention provides modified sophorolipids of the general formula (I), which is linked to one or more therapeutic agent cargos having alcohol, amine, carboxylic acid, and/or inorganic acid functionalization groups.

Thus, one aspect of the invention is a compound of the formula (I) or salts thereof:

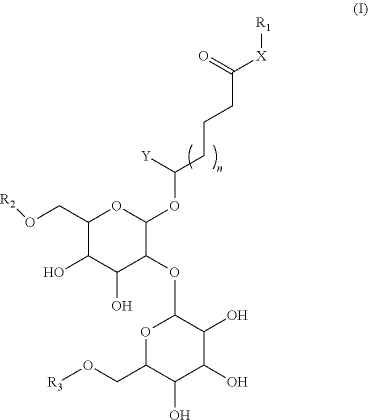

wherein:
Y is hydrogen or methyl;
R₁ is a first therapeutic agent cargo linked to the sophorolipid structure via an ester linkage with X═O or an amide linkage with X═NR₀, wherein R₀ is hydrogen or a substituent; or R₁ is hydrogen and X is O;
R₂ and R₃ are each independently hydrogen, acetyl, or a second therapeutic agent cargo linked to the sophorolipid structure via an ester linkage; and
n is an integer between 4 and 16 inclusively, preferably 12 when Y is methyl, and the chain defined by n has 0-3 unsaturation,
wherein at least one of R₁, R₂, and R₃ is a therapeutic agent cargo.

In a first set of embodiments, the present invention provides a compound of the formula (I) or salts thereof wherein:
Y is hydrogen or methyl;
R₁ is a first therapeutic agent cargo linked to the sophorolipid structure via an ester linkage with X═O or an amide linkage with X═NR₀, wherein R₀ is hydrogen or a substituent;
R₂ and R₃ are each independently hydrogen or acetyl; and
n is an integer between 4 and 16 inclusively, preferably 12 when Y is methyl, and the chain defined by n has 0-3 unsaturation.

In some embodiments, R₁ is a first therapeutic agent cargo that forms an ester linkage.

In other embodiments, R₁ is a first therapeutic agent cargo that forms an amide linkage with X═NR₀, wherein R₀ is hydrogen, lower alkyl, aryl, or a combination thereof.

According to a second set of embodiments, the present invention provides a compound of the formula (I) or salts thereof wherein:
Y is hydrogen or methyl;
R₁ is a first therapeutic agent cargo linked to the sophorolipid structure via an ester linkage with X═O or an amide linkage with X═NR₀, wherein R₀ is hydrogen or a substituent;
R₂ and R₃ are each independently hydrogen, acetyl, or a second therapeutic agent cargo linked to the sophorolipid structure via an ester linkage, provided that at least one of R₂ and R₃ is the second therapeutic agent; and
n is an integer between 4 and 16 inclusively, preferably 12 when Y is methyl, and the chain defined by n has 0-3 unsaturation, wherein the first and second therapeutic agents are the same or different compounds.

In some embodiments, R₁ is a first therapeutic agent cargo that forms an ester linkage.

In other embodiments, R₁ is a first therapeutic agent cargo that forms an amide linkage with X═NR₀, wherein R₀ is hydrogen, lower alkyl, aryl, or a combination thereof.

According to a third set of embodiments, the present invention provides modified sophorolipids of the formula (1) or salts thereof wherein:
Y is hydrogen or methyl;
R₁ is hydrogen and X is O;
R₂ and R₃ are each independently hydrogen, acetyl, or a second therapeutic agent cargo linked to the sophorolipid structure via an ester linkage, provided that at least one of R₂ and R₃ is the second therapeutic agent cargo; and
n is an integer between 4 and 16 inclusively, preferably 12, and the chain defined by n has 0-3 unsaturation.

The present invention covers all of the compounds represented by the general formula (I), including salts thereof, hydrates thereof, geometric and optical isomers thereof, and polymorphic forms thereof. Furthermore, it will be appreciated by a skilled artisan that the modified sophorolipid of formula (I) may exist in lactonic forms if the first therapeutic agent cargo at R₁ has a free acid such as carboxylic acid. That is, the free carboxylic acid can esterify internally with one of the hydroxyl substituents of the sophorose group.

According to some embodiments, the first therapeutic agent cargo for R₁ is salicylic acid, β-hydroxybutyric acid, amino acid, or succinic acid.

According to other embodiments, the second therapeutic agent cargo that can be R₂ or R₃ is independently salicylic acid, butyric acid, β-hydroxybutyric acid, amino acid, succinic acid, or phosphoric acid. The first and second therapeutic agent cargos may be same or different.

### Sophorolipids

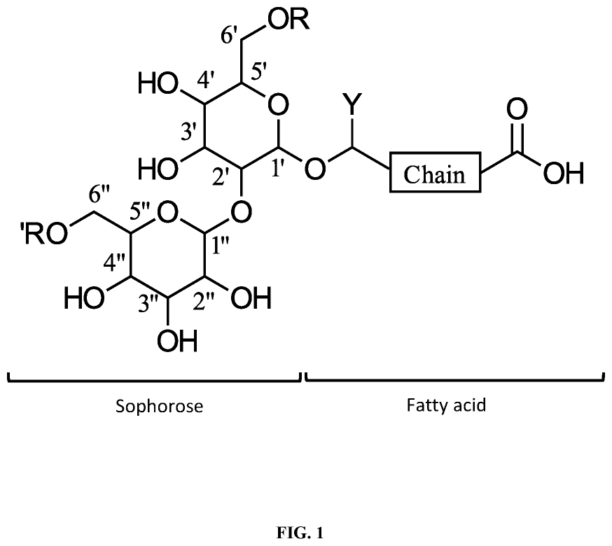

Sophorolipids are generally obtained from fermentations by microorganisms that use as carbon sources pure fatty acids, fatty acid mixtures, pure fatty acid esters, mixtures of fatty acid esters, triglycerides along with carbohydrate sources such as corn syrup, dextrins and glucose using a fermentation process comprising a wild-type or engineered yeast strain. As shown in FIG. 1, sophorolipids generally comprise a sophorose that may or may not be acetylated at the 6′ and 6″ positions and a hydroxylated fatty acid tail with various degrees of saturation and unsaturation. Most commonly, the fatty acids consist of 16 or 18 carbon atoms with one degree of unsaturation, but the fatty acid structure varies depending on the carbon sources provided during the production (e.g., degree of unsaturation, chain length) and the microorganisms conducting the fermentation.

The production of sophorolipids with the use of renewable substrates and different microbial species, as well as the variation in culture parameters (incubation time, stirring speed, pH of the medium and added nutrients), allow for the acquisition of compounds with distinct structural and physical properties. This makes it possible to produce a wide variety of compounds that can elicit different physical, chemical, biochemical, and biophysical properties.

The hydrophobic fatty acid tail of sophorolipids normally is terminally or sub-terminally linked glycosidically to the sophorose molecule. The fatty acid carboxylic acid group in sophorolipids is either free (acidic form as in FIG. 1) or intramolecularly esterified, generally at the 4″-position (lactonic form). The degree of lactonization of sophorolipids and acetylation of the sophorose portion may be influenced by, inter alia, the carbon source fed during sophorolipid production. For example, it has been found that sophorolipids derived from rapeseed, sunflower, and palm oils rich in C18:0 and C18:1 fatty acids are formed with higher levels of diacetylated lactones than sophorolipids produced from the corresponding fatty acid ester feedstocks.

Sophorolipids have environmental compatibility, high biodegradability, low toxicity, high selectivity, and specific activity in a broad range of temperature, pH, and salinity conditions.

### Fermentation

In some embodiments, the modified sophorolipids according to the present invention are prepared by chemically modifying sophorolipids that have been produced by a fermentation process known in the art in the presence of select fatty acids and carbohydrate feedstocks. As used herein “fermentation” refers to growth of cells under controlled conditions. The growth could be aerobic or anaerobic. The fermentation processes known in the art include, but are not limited to, solid-state fermentation, submerged fermentation, or modifications, hybrids and/or combinations thereof.

The sophorolipids according to the present invention can be derived via a fermentation process from a recombinant organism or by a strain that naturally produces sophorolipids. Non-limiting examples of sophorolipid-producing organisms include Candida bombicola, Candida apicola, Candida bogoriensis, Yarrowia lipolytica, Starmerella bombicola, Starmerella clade, Rhodotorula bogoriensis, Wickerhamiella domericqiae, and Wickerhamomyces anomalus. Some recombinant sophorolipid-producing microbes have been reported to allow control of sophorolipid structure. As a non-limiting example, certain recombinant S. bombicola strains may be utilized to produce either solely lactonic or solely acidic sophorolipids (see, e.g., Roelants et. al., Towards the Industrialization of New Biosurfactants: Biotechnological Opportunities for the Lactone Esterase Gene from Starmerella Bombicola, 113 Biotechnology and Bioengineering 3, 550-559 (2015)). In addition, a recombinant Candida bombicola strain with an acetyltransferase gene knockout can be used to produce sophorolipids without acetylation (see WO 2012/080116).

The microorganisms of interest can be cultivated at small or large scale on site and utilized, even being still-mixed with their media. The sophorolipids of the present invention can also be produced at large quantities at the site of need. The growth vessel used for growing sophorolipid-producing organisms can be any fermenter or cultivation reactor for industrial use.

In one embodiment, a single type of microorganism is grown in a reactor system. In alternative embodiments, multiple microorganisms, which can be grown together without deleterious effects on growth or the resulting product, can be grown in a single reactor system. There may be, for example, 2 to 3 or more different microorganisms grown in a single reactor at the same time. In some embodiments, more than one microorganism grows symbiotically in the reactor.

In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

The cultivation may be supplemented with a carbon source. The carbon source is typically a carbohydrate, such as glucose, sucrose, lactose, fructose, trehalose, mannose, mannitol, and/or maltose, preferably glucose; organic acids such as acetic acid, fumaric acid, citric acid, propionic acid, malic acid, malonic acid, and/or pyruvic acid; alcohols such as ethanol. isopropyl, propanol, butanol, pentanol, hexanol, isobutanol, and/or glycerol; fats and oils such as soybean oil, rice bran oil, canola oil, olive oil, corn oil, sesame oil, and/or linseed oil; etc. These carbon sources may be used independently or in a combination of two or more.

The cultivation can be supplemented with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The growing culture in the cultivation can also be oxygenated. One embodiment utilizes slow motion of air to remove low oxygen-containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride, and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the present invention include, but are not limited to, L-alanine, manganese, L-valine, and L-asparagine or any other known germination enhancer.

In some embodiments, additional acids and/or antimicrobials in the liquid medium before and/or during the cultivation process may be added. Antimicrobial agents or antibiotics are used for protecting the culture against contamination. Additionally, antifoaming agents may also be added to prevent the formation and/or accumulation of foam when gas is produced during cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the cultivation of microorganisms is carried out at about 5° C. to about 100° C., preferably, about 15° C. to about 60° C., more preferably, about 25° C. to about 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia, or other microbial propagules. In this manner, a quasi-continuous system is created.

In some embodiments, the sophorolipids produced by microorganisms of interest may be retained in the microorganisms or secreted into their growth medium. The sophorolipid content can be, for example, at least 20%, 30%, 40%, 50%, 60%, 70%, 80%, or 90%.

The growth medium may contain compounds that stabilize the activity of the sophorolipids. The sophorolipids can be purified, or the sophorolipids can be used in crude form, meaning they are not separated from the fermentation broth in which they were produced.

In certain embodiments, the sophorolipid is isolated and/or purified from the growth medium resulting from fermentation of a biosurfactant-producing microorganism. Isolation and purification can be easily achieved using standard methods or techniques described in the literature. The sophorolipid can be further concentrated, if desired.

As used herein, the terms “isolated” or “purified,” when used in connection with biological or natural materials such as glycolipids means the material is substantially free of other compounds, such as cellular material, with which it is associated in nature. That is, the materials do not occur naturally without these other compounds and/or have different or distinctive characteristics compared with those found in the native material.

In certain embodiments, purified compounds are at least 60% by weight of the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

### Modification Methods

In some embodiments, the modified sophorolipids according to the present invention are obtained by chemically modifying sophorolipids that have been obtained from the product of fermentation by sophorolipid-producing microbes. The sophorolipids to be chemically modified may be obtained by fermentation methods and processes as described above and/or any other methods known in the art. Any suitable techniques and chemical reactions known in the art may be used to modify sophorolipids. For example, a therapeutic agent cargo may be added at the R₁, R₂, and/or R₃ position of formula (I) via an esterification reaction. Similarly, amide coupling reactions known in the art may be utilized to form an amide bond R₁—X—CO—in formula (I) wherein X is NR₀.

As an example, Fischer esterification is a commonly known esterification reaction characterized by the combining of an alcohol and an acid (with acid catalysis) to yield an ester plus water. Esterification reaction can also be carried out with inorganic acids under appropriate conditions with specialized reagents. It may be necessary to introduce one or more protecting group to improve chemoselectivity for the desired esterification reaction.

Chemical modifications can also be made, for example, to alter the degree of unsaturation on the fatty acid chain. Commonly known hydrogenation or dehydrogenation reactions, or addition or elimination reactions may be used.

### Therapeutic Agent Cargo

A therapeutic agent cargo as used herein can be any suitable molecule having a hydroxyl, carboxyl, inorganic acid, and/or amine group such that it is capable of forming an ester or amide linkage with a sophorolipid structure at the defined linkage points in formula (I). In certain embodiments, the therapeutic agent cargo is capable of being released from the sophorolipid structure to impart its cosmetic, therapeutic, or pharmaceutical benefits at the target site. By way of example, the therapeutic agent cargo may be released by hydrolysis and/or by the action of enzymes such as lipases.

In certain embodiments, suitable molecules for the therapeutic agent cargo for R₁ have at least one alcohol group such that it is capable of forming an ester linkage, or an amine of the formula —NR₀H wherein R₀ is hydrogen or a substituent, preferably hydrogen, lower alkyl, aryl, or a combination thereof, such that it is capable of forming an amide linkage with the sophorolipid structure in formula (1). Non-limiting examples of molecules suitable for the therapeutic agent cargo for R₁ include salicylic acid, β-hydroxybutyric acid, amino acid, and/or succinic acid.

Similarly, suitable molecules for the therapeutic agent cargo for R₂ and R₃ will typically have at least one carboxylic acid or inorganic acid group such that it is capable of forming an ester linkage with the sophorolipid structure. Non-limiting examples of molecules suitable for the therapeutic agent cargo for R₂ and R₃ include salicylic acid, butyric acid, β-hydroxybutyric acid, amino acid, succinic acid, and/or phosphoric acid. If both R₂ and R₃ of formula (I) are therapeutic agent cargos, they may be same or different. Preferably, R₂ and R₃ are the same molecule if they are both therapeutic agent cargos.

According to certain embodiments of the invention, at least one of R₁, R₂, and R₃ is a therapeutic agent cargo. If both R₂ and R₃ are therapeutic agent cargos, they are preferably the same molecules. If R₁ and at least one of R₂ and R₃ are therapeutic agent cargos, the therapeutic agent cargos are the same or different. It would be appreciated by a skilled artisan that, even if they are the same molecule, however, the points of attachment to the sophorolipid structure may differ in formula (I). For example, the point of attachment for salicylic acid at R₁ will be the hydroxyl group, while its point of attachment at R₂ or R₃ will be carboxylic acid group (see Table 1 below).

TABLE 1
Exemplary points of attachment (represented with wavy lines) in
salicylic acid at R₁ and R₂/R₃

| R₁ | R₂ or R₃ |
|---|---|
| 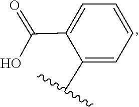 [SMILES: *c1ccccc1C(=O)O] | 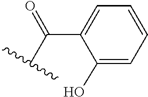 [SMILES: *C(=O)c1ccccc1O] |
| X = O | |

In preferred embodiments, therapeutic agent cargo is a pharmaceutically or cosmetically active ingredient. Non-limiting examples of therapeutic agent cargos include, but are not limited to, natural enzymes, proteins derived from natural sources, recombinant proteins, natural peptides, synthetic peptides, cyclic peptides, antibodies, cytotoxic agents, immunoglobins, beta-adrenergic blocking agents, calcium channel blockers, coronary vasodilators, cardiac glycosides, antiarrhythmics, cardiac sympathomimetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thiazolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones. bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT4 partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A2 inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptase inhibitors, protease inhibitors, anemia, Kaposi's sarcoma, aminoglycosides, carbapenems, cephalosporins, glycopeptides, lincosamides, macrolies, oxazolidinones, penicillins, streptogramins, sulfonamides, trimethoprim and derivatives, tetracyclines, anthelmintics, amebicides, biguanides, cinchona alkaloids, folic acid antagonists, quinoline derivatives, Pneumocystis carinii therapy, hydrazides, imidazoles, triazoles, nitroimidazoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigraine preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, anthracyclines, actinomycins, camptothecin derivatives, epipodophyllotoxins, taxanes, vinca alkaloids and analogs, antiandrogens. antiestrogens, nonsteroidal aromatase inhibitors, protein kinase inhibitor antineoplastics, azaspirodecanedione derivatives, anxiolytics, stimulants, monoamine reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g., lidocaine, articaine hydrochloride, bupivacaine hydrochloride), antipyretics, hypnotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opioid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists, and/or derivatives and analogs of any of the foregoing molecules.

Preferably, the therapeutic agent cargo according to the present invention is a small molecule drug. Typically, small molecule drugs have molecular weights under 1,000 Daltons. They tend to be chemically synthesized, but in some cases are purified natural products.

### Amino Acids

In some embodiments, the therapeutic agent cargo according to the invention is an amino acid. The amino acid as used herein may be L.-amino acid or D-amino acid. It can be natural amino acids (such as essential amino acids, non-essential amino acids, or conditional amino acids) or synthetic/modified amino acids that do not exist in nature. Non-limiting examples of amino acids include alanine, β-alanine, arginine, asparagine, aspartic acid, cysteine, homocysteine, glutamic acid, glutamine, glycine, histidine, isoleucine, leucine, lysine, methionine, phenylalanine, proline, serine, taurine, homotaurine, threonine, tryptophan, tyrosine, valine, theanine, D-2-amino-3-guanidinopropionic acid, GABA, citrulline, tranexamic acid, aminocaproic acid, 4-amino-5-hexenoic acid, 4-oxaproline, 4-thioproline, 2-azaproline, 4-hydroxyproline, 1,5-disubstituted tetrazole, 2-amino isobutyric acid, sarcosine, 1-aminocyclopentane-1-carboxylic acid, beta alanine, 2-amino-cyclopentane carboxylic acid (beta-proline), 5-hydroxylysine, hydroxylysine-5-sulfate, hydroxylysine-5-nitrate, hydroxylysine-5-phosphate, serine-3-sulfate, threonine-3-sulfate, serine-3-nitrate, threonine-3-nitrate, serine-3-phosphate, threonine-3-phosphate, 2-hydroxy alkanoic acid, 5-aminopentanoic acid, and 5-amino-2-oxopentanoic acid. Preferred amino acids are glutamine. glutamic acid, arginine, glycine, lysine, threonine, and sulfur-containing amino acids such as methionine, cysteine, homocysteine, and taurine.

Amino acids can be included in various cosmetic or pharmaceutical products such as creams, lotions, soaps, cosmetics, and hair care products. As a moisturizer, many amino acids are thought to be critical to the natural moisturizing factor (NMF) of the stratum corneum of the skin and also to retain moisture in the hair. Roughly half of the NMF of skin is amino acids and pyrrolidone carboxylic acid (pyroglutamic acid or PCA), which is derived from glutamic acid or glutamine. Various amino acids are building blocks for collagen, promote wound healing as well as smooth fine lines.

Dietary supplementation of amino acids, in particular essential amino acids, has been shown to promote general well-being. Amino acids regulate glucose and lipid metabolism and energy balance, increase mitochondrial biogenesis, and maintain immune homeostasis. Dietary amino acids are also major fuels for the small intestinal mucosa, as well as important substrates for syntheses of intestinal proteins, nitric oxide, polyamines, and other products of biological importance. Studies support potential therapeutic roles for amino acids including glutamine, glutamic acid, arginine, glycine, lysine, threonine, and sulfur-containing amino acids in gut-related diseases. For example, ingestion of glutamine has been shown to improve gut health by supporting the gut microbiome, gut mucosal wall integrity, and by modulating inflammatory responses.

### Butyric Acid/Butyrate

In some embodiments, the therapeutic agent cargo according to the invention is butyric acid. Butyric acid/butyrate belongs to a group of short-chain fatty acids. It is thought to play several beneficial roles in, inter alia, the gastrointestinal tract. Butyrate is easily absorbed by enteric cells and used as a main source of energy. Butyrate has been shown to be a key fuel for epithelial cells of the intestinal tract and that it may improve intestinal regulatory T-cells as well as gut lining integrity.

Furthermore, butyric acid has been shown to regulate colonocyte proliferation and apoptosis, gastrointestinal tract motility, and bacterial microflora composition. Luminal butyrate adversely affects pathogenic gram-negative bacteria such as Escherichia coli, Salmonella spp., and Campylobacter spp. Butyrate is shown to reduce or inhibit the microbiome population responsible for generating propionic acid, which is implicated in Autism Spectrum Disorders.

Butyric acid also plays a beneficial role in other biological processes including insulin sensitivity, neurological processes, immunoregulation and anti-inflammatory activity. As an example, orally consumed butyrate is shown to induce GLP-1 secretion, a hormone known to support the improvement of glucose tolerance and appetite control. Orally administered butyrate has been observed to exert an anti-inflammatory effect, attenuating experimentally induced colitis and causing remission of Crohn's disease through downregulation of NF-KB and IL-1β. It has also been suggested to stimulate neurogenesis in the ischemic brain via brain derived neurotrophic factor (BDNF) upregulation, generate antidepressant-like effects, prevent cardiovascular health, and lower endogenous cholesterol production.

β-hydroxybutyric Acid/β-hydroxybutyrate

In other embodiments, the therapeutic agent cargo according to the invention is β-hydroxybutyric acid. β-hydroxybutyric acid is naturally synthesized in the liver from fatty acids and is an essential carrier of energy from the liver to peripheral tissues when the supply of glucose is too low. β-hydroxybutyric acid also has cellular signaling functions.

Therapeutic effects of β-hydroxybutyric acid have been implicated in multiple disease models. For example, an in vivo study showed that β-hydroxybutyrate supplementation decreased tumor growth and prolonged subject survival independent of other dietary inclusions. β-hydroxybutyrate is also shown to play a role in mediating inflammation, suggesting potential application in the treatment of autoinflammatory diseases. It has also been shown to impede brain hypometabolic diseases like Alzheimer's disease (AD) and seizure onset in epilepsy and to help improve cardiac health by reducing myocardial glucose uptake and increasing blood flow.

β-hydroxybutyric acid has been linked to improved gut health. Administration of β-hydroxybutyric acid in the form of D-β-hydroxybutyrate (DBHB) has been shown to increase production of butyrate among DBHB-utilizing strains among human colonic microbiota, including the genus Coprococcus. Human colonic microbiotas with high growth activity demonstrate efficient utilization of DBHB for increased butyrate production, which affords health benefits. As discussed above, colonic generation of butyrate has beneficial effects on human health, and growth. Moreover, recent review paper suggests potential synergies of β-hydroxybutyrate and butyrate on the modulation of metabolism, inflammation, cognition, and general health. Cavaleri, F. and Bashar, E., Potential Synergies of β-Hydroxybutyrate and Butyrate on the Modulation of Metabolism, Inflammation, Cognition, and General Health, J. Nutrition and Metabolism, Article ID 7195760 (2018).

### Salicylic Acid/Salicylate

In some embodiments, the therapeutic agent cargo according to the invention is salicylic acid. Salicylic acid is a beta hydroxy acid that occurs as a natural compound in plants. It has direct activity as an anti-inflammatory agent and acts as a topical antibacterial agent as well as promotes exfoliation. One exemplary use of salicylic acid is treatment of acne.

Salicylic acid is the principal metabolite of aspirin, which is an acetylated salicylic acid. Aspirin is used as a medication to reduce pain, fever, or inflammation. Specific inflammatory conditions which aspirin is used to treat include, but are not limited to, Kawasaki disease, pericarditis, and rheumatic fever.

### Succinic Acid/Succinate

In other embodiments, the therapeutic agent cargo according to the invention is succinic acid/succinate. Succinate is a metabolic intermediate of the tricarboxylic acid (TCA) cycle within host cells. Succinate is also produced in large amounts during bacterial fermentation of dietary fiber. Succinic acid has generally been shown to be a “natural” antibiotic because of its relative acidity and safety. Recently, succinic acid has been suggested as an active ingredient for treating acne in topical formulations. Succinate has been provided as supplements to help reduce the effects of hangovers by facilitating the degradation of acetaldehyde, a toxic byproduct of alcohol metabolism. Succinic acid has been shown to stimulate neural system recovery and bolster the immune system.

### Phosphoric Acid/Phosphate

Phosphoric Acid, also known as orthophosphoric acid or phosphoric (V) acid, occurs as a white solid or as a colorless, viscous liquid. Phosphate is typically the drug form of phosphorous that can be ingested. While the body cannot produce phosphorus on its own, it is an essential nutrient for all living things. For example, phosphorus plays an important role in facilitating effective digestion in the human body. Low levels of phosphorus in the body have been linked to various ailments including poor gut health, as phosphorus is an essential element of bacterial survival and reproduction. A study on phosphorus supplementation in broiler chickens demonstrated an increase in the abundance of the butyrate-producing bacteria Faecalibacterium and Pseudoflavonifractor in the cecal digesta. A dietary intervention study in human with phosphorus supplementation (1000 mg/day) showed that fecal microbial diversity improved, and short chain fatty acids concentration increased. Yang, Q. et al., Role of Dietary Nutrients in the Modulation of Gut Microbiota: A Narrative Review, 12 Nutrients 381, at 6 (2020). Short chain fatty acids such as butyrate, as described above, play an important role in gut and metabolic health.

### Pharmaceutically or Cosmetically Acceptable Salts

In some embodiments, the modified sophorolipids of the present invention are provided in the form of a pharmaceutically or cosmetically acceptable salt. In specific embodiments, a carboxylate salt is formed when X is O⁻ and R₁ is a cationic fragment such that there is no covalent bond between X and R₁ in formula (I). The therapeutic agent cargos may also form a corresponding salt if it contains an appropriate functional group. Salts may be formed by procedures well known and described in the art.

Examples of pharmaceutically acceptable cations for salts include, without limitation, aluminium, arginine, benzathine, calcium, chloroprocaine, choline, diethanolamine, ethanolamine, ethylenediamine, lysine, magnesium, histidine, lithium, meglumine, potassium, procaine, sodium, triethanolamine, and zinc. Preferred cations for R₁ when X is O⁻ are lithium (Li⁺), sodium (Na), and potassium (K⁺), more preferably sodium (Na⁺), and potassium (K⁺).

Depending on the chemical characteristics of the therapeutic agent cargos in the modified sophorolipids, acid addition salts may be formed. Examples of pharmaceutically acceptable addition salts include, but are not limited to, the non-toxic inorganic and organic acid addition salts such as the acetate derived from acetic acid, the aconate derived from aconitic acid, the ascorbate derived from ascorbic acid, the benzenesulfonate derived from benzenesulfonic acid, the benzoate derived from benzoic acid, the cinnamate derived from cinnamic acid, the citrate derived from citric acid, the embonate derived from embonic acid, the enantate derived from enanthic acid, the formate derived from formic acid, the fumarate derived from fumaric acid, the glutamate derived from glutamic acid, the glycolate derived from glycolic acid, the hydrochloride derived from hydrochloric acid, the hydrobromide derived from hydrobromic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the nitrate derived from nitric acid, the perchlorate derived from perchloric acid, the phosphate derived from phosphoric acid, the phthalate derived from phthalic acid, the salicylate derived from salicylic acid, the sorbate derived from sorbic acid, the stearate derived from stearic acid, the succinate derived from succinic acid, the sulphate derived from sulphuric acid, the tartrate derived from tartaric acid, the toluene-p-sulphonate derived from p-toluene sulphonic acid, and the like.

Other salts which may not be considered pharmaceutically acceptable, may be useful in the preparation of salts useful as intermediates in obtaining a modified sophorolipid of the invention and its pharmaceutically acceptable salt.

In another embodiment, the modified sophorolipids of the invention are used in their respective acid form according to the present invention.

The modified sophorolipids of the invention may be provided in unsolvated or solvated forms together with a pharmaceutically acceptable solvent(s) such as water, ethanol, and the like. Solvated forms may also include hydrated forms such as the monohydrate, the dihydrate, the hemihydrate, the trihydrate, the tetrahydrate, and the like.

### Compositions

Another aspect of the invention is directed to cosmetic or pharmaceutical compositions comprising the modified sophorolipid of the present invention as an active, administered to a subject.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

The particular mode of administration as well as the dosage regimen designed to deliver an effective amount of the modified sophorolipids are appropriately decided, taking into account factors such as the nature of the therapeutic agent cargo(s), age, sex, and particulars of the subject, as well as the condition, the disease, and the disease state involved, and whether the purpose is preventative. As used herein, the terms “effective amount,” and “effective dose” are used to refer to an amount of something (e.g., a compound, a composition, time) is capable of causing a desired outcome (e.g., improvement in skin heath and/or appearance). Administration of an effective amount of the modified sophorolipids may be accomplished in daily or multi-daily doses of the modified sophorolipids over a period of a few days to months, or even years. If the modified sophorolipids is administered with another therapeutic agent, the effective amount of the modified sophorolipids may carry the therapeutic agent cargo(s) in the same range as is typical for use of that agent as a monotherapy, or the amount may be lower than a typical monotherapy amount especially if the combination therapy results in a synergy.

The composition according to the present invention may comprise an isolated and/or purified form of the modified sophorolipid as an active ingredient. In other embodiments, the modified sophorolipids in a composition according to the present invention are provided in crude form, i.e., in the fermentation broth in which they were produced. The crude form can comprise, for example, at least 20%, 30%, 40%, 50%, 60%, 70%, 80% or 90% w/v of the modified sophorolipids in broth.

The composition may further include additional active or inactive ingredients, suitable for the mode of administration and intended purpose, provided that such addition does not adversely interfere with the functions of the modified sophorolipids.

In yet other embodiments, acceptable carriers may be included in the composition of the present invention, depending on the form of the composition and/or mode of administration. Pharmaceutically or cosmetically acceptable carries include but are not limited to, pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid, or talc. If desired or suitable, a coating material may also be used such as glyceryl monostearate or glyceryl distearate, for example, to delay absorption in the gastrointestinal tract if appropriate and the pharmaceutical composition is in the form of a solid form.

In certain embodiments, the composition according to the present invention further comprises microbial growth by-products. Particularly, embodiments of the present invention provide a topical therapeutic composition for promoting the healing of skin conditions or an ingestible composition for improving gut health, wherein the composition comprises therapeutically effective amounts of one or more biochemicals produced by the cultivation of microorganisms.

In certain embodiments, the microbial growth by-products are amphiphilic molecules, enzymes and/or proteins. In one embodiment, the microbial growth by-products have antimicrobial and/or anti-biofilm properties. In one embodiment, the microbial growth by-products have skin rejuvenating properties, meaning they help make skin appear younger, smoother, and brighter.

In certain embodiments, the composition further comprises one or more amphiphilic molecules, wherein the amphiphilic molecules are biosurfactants selected from, for example, low molecular weight glycolipids (e.g., sophorolipids, rhamnolipids, mannosylerythritol lipids and trehalose lipids), lipopeptides (e.g., surfactin, iturin, fengycin, athrofactin and lichenysin), cellobiose lipids, flavolipids, phospholipids (e.g., cardiolipins), and high molecular weight polymers such as lipoproteins, lipopolysaccharide-protein complexes, and polysaccharide-protein-fatty acid complexes. The amphiphilic molecules may be a modified form, derivative, fraction, isoform, isomer, or subtype of a biosurfactant, including forms that are biologically or synthetically modified. In one embodiment, the one or more biosurfactants are present in the composition in critical micelle concentration (CMC).

The composition can be placed in containers of appropriate size, taking into consideration, for example, the intended use, the contemplated method of application, the size of the fermentation vessel, and any mode of transportation from microbe growth facility to the location of use. Thus, the containers into which the composition is placed may be, for example, from 0.1 gallon to 1,000 gallons or more. The composition may further be placed into smaller containers, such as bottles, for distribution of individual doses of the composition.

The composition according to the present invention may come in various physical forms. In some embodiments, the composition is in the form of solids including tablets, filled capsules, powder and pellet forms. In another embodiment, the pharmaceutical composition may be in the powder form, in which the pharmaceutically accepted carrier is a finely divided solid that is in a mixture with the finely divided active ingredient. In a further embodiment, the pharmaceutical composition according to the present invention is a sustained release system such as semipermeable matrices of solid hydrophobic polymers containing the modified sophorolipids of the present invention. In another embodiment, the pharmaceutical composition is in a liquid form such as aqueous or non-aqueous solutions, suspensions, emulsions, elixirs, and capsules filled with the same.

Any appropriate mode of administration can be utilized to administer the compositions comprising the modified sophorolipids according to the present invention. Such mode includes, but is not limited to, oral, rectal, bronchial, nasal, topical, buccal, sub-lingual, transdermal, vaginal, intramuscular, intraperitoneal, intravenous, intra-arterial, subcutaneous, intracerebral, intraocular administration or in a form suitable for administration by inhalation or insufflation, including powders and liquid aerosol administration, or intraparenteral infusion. Administration may be also by way of other carriers or vehicles such as patches, micelles, liposomes, vesicles, implants (e.g., microimplants), synthetic polymers, and the like. Factors that determine the mode of administration of a particular composition include the nature of the therapeutic agent cargo that is being delivered to the subject. In some embodiments, the mode of administration is oral or rectal. In other embodiments, the mode of administration is topical.

Orally administered compositions according to the invention are any preparations or compositions suitable for consumption, for nutrition, for oral hygiene and/or for pleasure, and are products intended to be introduced into the human or animal oral cavity, to remain there for a certain period of time and then to either be swallowed (e.g., food ready for consumption) or to be removed from the oral cavity again (e.g., chewing gums or products of oral hygiene or medical mouth washes). These products include all substances or products intended to be ingested by humans or animals in a processed, semi-processed or unprocessed state. This also includes substances that are added to orally consumable products (particularly food and pharmaceutical products) during their production, treatment or processing and intended to be introduced into the human or animal oral cavity.

In certain embodiments, the composition can further comprise components that are, for example, sources of energy, nutrients and/or other health-promoting supplements, flavorings, preservatives, pH adjusters, sweeteners and/or dyes. The composition may also be carbonated. In one embodiment, the composition is formulated as a dehydrated powder or concentrate that can be reconstituted into a drinkable fluid by the addition of water. In one embodiment, the composition is formulated as a blended smoothie or milkshake.

Orally administered compositions can also include substances intended to be swallowed by humans or animals and then digested in an unmodified, prepared, or processed state; the orally administered compositions according to the invention therefore also include casings, coatings or other encapsulations that are intended also to be swallowed together with the product or for which swallowing is to be anticipated.

In other embodiments, the compositions according to the present invention are formulated for direct administration into the GI tract. For example, the composition can be formulated for administration to the proximal lower GI by hand or colonoscopy, to the distal lower GI tract via enema or rectal tubes, and the upper GI tract via nasogastric tubes, duodenal tubes, and endoscopy/gastroscopy.

In certain embodiments, the present invention provides topical skin care compositions comprising one or more of the modified sophorolipids according to the present invention. In some embodiments, the topical composition can further comprise a dermatologically acceptable carrier, and one or more active or inactive cosmetic ingredients, such as, e.g., vitamins, moisturizers, dyes, fragrances, sunscreens, exfoliants, essential oils, botanical extracts, and so on.

The dermatologically acceptable carrier may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane and isohexadecane; silicone oils such as cyclomethicone, dimethicone, dimethicone cross-polymer, polysiloxanes and their derivatives, preferably organo-modified derivatives including PDMS.

dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous carriers may include one or more solvents miscible with water, including lower alcohols, such as ethanol, isopropanol, and the like. The carrier may comprise from about 1% to about 99% by weight of the composition, from 10% to about 85%, from 25% to 75%, or from 50% to about 65%.

Non-biological surfactants can also be added to the formulation. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropyl betaine), sultaines, sacrosinates, isethionates, taurates, ethoxylated sorbitan esters, alkanolamides and amino acid-based surfactants.

Viscosity modifiers can also be added to the compositions, including, for example, cocamide DEA, oleamide DEA, sodium chloride, cellulosic polymers, polyacrylates, ethoxylated esters, alcohol, glycols, xylene sulfonates, polysorbate 20, alkanolamides, and cellulose derivatives (e.g., hydroxypropyl methylcellulose and hydroxyethyl cellulose).

Polymers can also be added, including, for example, xanthan gum, guar gum, polyquaternium-10, PEG-120 methyl glucose dioleate, PEG-150 distearate, PEG-150 polyglyceryl-2 tristearate and PEG-150 pentaerythrityl tetrastearate.

In one embodiment, the composition may include additional skin actives, including but not limited to, keratolytic agents, desquamating agents, keratinocyte proliferation enhancers, collagenase inhibitors, elastase inhibitors, depigmenting agents, anti-inflammatory agents, steroids, anti-acne agents, antioxidants (e.g., ascorbic acid), advanced glycation end-product (AGE) inhibitors, exfoliating agents (e.g., glycolic acid, 3,6,9-trioxaundecanedioic acid, etc.), estrogen synthetase stimulating compounds (e.g., caffeine and derivatives), compounds capable of inhibiting 5 alpha-reductase activity (e.g., linolenic acid, linoleic acid, finasteride, and mixtures thereof), barrier function enhancing agents (e.g., ceramides, glycerides, cholesterol and its esters, alpha-hydroxy and omega-hydroxy fatty acids and esters thereof), retinoids, sunscreen, to name a few.

The composition may optionally comprise additives, adjuvants, or other components of topical compositions known to those skilled in the art including, but not limited to: skin penetration enhancers; emollients (e.g., isopropyl myristate, petrolatum, volatile or non-volatile silicones oils, such as methicone and dimethicone, ester oils, mineral oils, and fatty acid esters); humectants (e.g., glycerin, hexylene glycol, caprylyl glycol); skin plumpers (e.g., palmitoyl oligopeptide, collagen, collagen and/or glycosaminoglycan (GAG) enhancing agents); anti-inflammatory agents (e.g., Aloe vera, bioflavonoids, diclofenac, salicylic acid); chelating agents (e.g., EDTA or a salt thereof, such as disodium EDTA); vitamins (e.g., tocopherol and ascorbic acid); vitamin derivatives (e.g., ascorbyl monopalmitate, tocopheryl acetate, Vitamin E palmitate); thickeners (e.g., hydroxyalkyl cellulose, carboxymethylcellulose, carbombers, and vegetable gums, such as xanthan gum); gelling agents (e.g., ester-terminated polyester amides); structuring agents; proteins (e.g., lactoferrin); immune modulators (e.g., corticosteroids and non-steroidal immune modulators). Further examples include, but are not limited to: stabilizers; fragrances; film formers; insect repellents; skin cooling compounds; skin protectants; lubricants; preservatives; pearls; chromalites; micas; conditioners; anti-allergenics; pH adjusters; and antimicrobials.

The composition may be formulated as a suspension, emulsion, hydrogel, multiphase solution, vesicular dispersion or in any other known form of topical skin composition.

In certain embodiments, the topical composition may be formulated so that it can be applied, for example, via pen, tube, bottle, brush, stick, sponge, cotton swab, towelette (wipe), sprayer, dropper, hand, or finger.

The topical composition may be formulated in a variety of product forms, such as, for example, a lotion, cream, serum, spray, aerosol, liquid cake, ointment, essence, gel, paste, patch, pencil, powder, towelette, soap or other cleanser, shampoo, conditioner, stick, foam, mousse, elixir or concentrate.

In one embodiment, the composition can be provided in the form of a facial mask. The mask may be a peel-off mask or wash-off mask comprising the composition of the present invention.

In other embodiments, the composition of the present invention can be provided in a wound dressing, bandage, or a patch that may be applied, attached, or coupled to one or more layers of the skin or tissue of the subject. For example, the composition may be applied to a wound dressing, bandage, or a patch, which can then be placed over the area of skin being treated.

A wound dressing, bandage, and a patch can be made, for example, from any material that is dermatologically acceptable and suitable for placing on a wound or the skin. In exemplary embodiments, the A wound dressing, bandage, and a patch may be made from a woven or non-woven fabric of synthetic or non-synthetic fibers, or any combination thereof. The dressing may also include a support, such as a polymer foam, a natural or man-made sponge, a gel or a membrane that may absorb or have disposed thereon, a composition. By way of example, the support can be a film, a natural or synthetic polymer, or a rigid or malleable material (e.g., gauze). The wound dressing, bandage, and a patch may be absorbent and can be, for example, wetted with a composition of the present invention before applying the gauze to a wound, scar or other sites. In one embodiment, the wound dressing, bandage, or a patch may be impregnated with a composition of the subject invention and, optionally, dried. This allows the impregnated material to be stored for later use, or to avoid excessively dampening of the skin.

In other embodiments, the pharmaceutical composition may be formulated for parenteral administration (e.g., by injection, for example bolus injection or continuous infusion). In addition, the composition may be presented in unit dose form in ampoules, pre-filled syringes, and small volume infusion or in multi-dose containers with or without an added preservative. The composition may be in forms of suspensions, solutions, or emulsions in oily or aqueous carriers. The composition may further contain formulation agents such as suspending, stabilizing and/or dispersing agents. In a further embodiment, the active ingredient of the composition according to the invention may be in a powder form, obtained by aseptic isolation of sterile solid or by lyophilization from solution for constitution with a suitable carrier, e.g., sterile, pyrogen-free water, before use.

In yet other embodiments, the pharmaceutical composition may be formulated for direct administration to the nasal cavity by conventional means, for example with a dropper, pipette, or spray. The compositions may be provided in single or multi-dose form. Administration to the respiratory tract may also be achieved by means of an aerosol formulation in which the active ingredient is provided in a pressurized pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin.

Methods of Improving Bioavailability and/or Dermal Penetrability

Another aspect of the present invention is directed to a method of improving the bioavailability and/or dermal penetrability of an active ingredient in a subject by administering to the subject an effective amount of a modified sophorolipid of formula (I) as described herein. In particular, the modified sophorolipid of the present invention is capable of carrying at least one active ingredient (i.e., therapeutic agent cargo), wherein the active ingredient is linked to a sophorolipid structure via an ester or an amide bond. The suitable therapeutic agents as used herein have a chemical structure with at least one of alcohol, amine, carboxylic acid, and inorganic acid functional groups that are capable of forming an ester or amide linkage with a sophorolipid at the defined linkage points of formula (I) as described herein.

According to certain embodiments of the invention, the modified sophorolipids of formula (I) that are chemically linked to an active ingredient (i.e., therapeutic agent cargo) via an ester or amide bond are able to enhance and amplify the beneficial effects of the active ingredient as well as the sophorolipid. Specifically, the modified sophorolipids of formula (I) linked to an active ingredient (i.e., therapeutic agent cargo) contributes to more efficient and successful facilitation of absorption and/or dermal penetration of the active ingredient by the target body, compared to the active ingredient that is not linked to a sophorolipid structure. Without being bound by theory, bioavailability and/or dermal penetrability is enhanced by, for example, suppressing P-glycoproteins and/or modulating other physical barrier mechanisms that would otherwise reduce the penetration of certain active ingredients into, for example, a subject's epithelial cells and/or across the blood-brain barrier (BBB). Additionally, or alternatively, bioavailability and/or dermal penetrability may be enhanced by protecting the active ingredient from degradation either during storage or in vivo. Additionally, or alternatively, bioavailability may be enhanced by improving the solubility of the active ingredient because of its linkage to the sophorolipid structure, either in a composition prior to administration or in vivo after administration.

Depending on the nature and effects of the therapeutic agent cargo being carried by the sophorolipid structure, the modified sophorolipids of the present invention are able to treat or prevent various diseases, ailments, and/or conditions.

As used herein, the term “treatment” refers to eradicating, reducing, ameliorating, or reversing, a degree, sign or symptom of a condition or disorder to any extent, and includes, but does not require, a complete cure of the condition or disorder. Treating can be curing, improving, or partially ameliorating a disorder.

As used herein, “preventing” a condition or disorder refers to avoiding, delaying, forestalling, or minimizing the onset of a particular sign or symptom of the condition or disorder. Prevention can, but is not required to be, absolute or complete, meaning the sign or symptom may still develop at a later time. Prevention can include reducing the severity of the onset of such a condition or disorder, and/or inhibiting the progression of the condition or disorder to a more severe condition or disorder. For example, in one embodiment, preventing hyperpigmentation can refer to avoiding, delaying, forestalling, or minimizing one or more unwanted features associated with skin hyperpigmentation, such as reducing the darkness or size of hyperpigmented areas that eventually develop. As another example, in one embodiment, preventing acne can refer to avoiding, delaying, forestalling, or minimizing one or more unwanted features associated with acne, such as reducing the number, darkness, and/or size of comedones that eventually develop, lessening the severity of acne that eventually develops, and/or completely or almost completely preventing the growth of P. acnes, the development of acne blemishes, and the other symptoms of acne.

### Method of Treating Skin Conditions or Ailments

In some embodiments, the present invention is directed to a method of treating skin conditions or ailments with the modified sophorolipid of formula (I) as described herein. As used herein, the term “skin condition” or “skin ailments” encompass any human and animal conditions, disorders, or diseases affecting the epidermis, dermis (including connective tissue, sebaceous glands, and hair follicles), and/or the subcutaneous tissue (hypodermis). Skin conditions that can, in certain embodiments, be treated and/or prevented using compositions, products and methods described herein include, but are not limited to, wounds (including, e.g., burns), scars, acne, blemishes, rosacea, folliculitis, carcinoma, melanoma, perioral dermatitis, cellulitis, carbuncles, photodamage, skin aging (e.g., wrinkles and dryness), age spots, psoriasis, ichthyosis, atopic dermatitis, rashes (including but not limited to erythematosus, macular, papular and/or bullous conditions), chronic wounds, bed sores, keratosis pilaris, sebaceous cysts, vitiligo, melisma, warts, inflammatory dermatoses, allodynia, ectopic dermatitis, telangiectasia, post-inflammatory hyperpigmentation, keratoses, eczema, xerosis, pruritis, lichen planus, nodular prurigo, microbial infection, body odor, scalp conditions and miliaria. Symptoms of skin conditions can include, for example, skin irritation/sensitivity, blemishes and other acneiform symptoms, pigmentation, or loss thereof, flushing, inflammation, wrinkles, dryness, sagging, thickening, scaling, scarring, flaking, rash, hives, blisters, ulcers, pecling, hair loss and other changes in the health, function, and appearance of the skin. In some embodiments, a symptom of a skin condition can also be a skin condition itself.

In some embodiment, the present invention is directed to a method of treating or preventing acne, or acne vulgaris. Acne results from the action of hormones and other impurities at the skin's oil glands and hair follicles. Acne is a systematic inflammatory disease resulting from blockage of sebaceous glands and hair follicles, which can become infected by the bacterium Propionibacterium acnes (P. acnes). Acne as used herein also includes comedonal acne (or “comedones”), which causes bumpy skin, blackheads, and non-inflamed blemishes.

In certain embodiments, the method of treating acne comprises topically administering to a subject an effective amount of the modified sophorolipid of formula (I) having at least one of salicylic acid and/or succinic acid as a therapeutic agent cargo, such as compound nos. 1-5, 19-24 (Examples). In other embodiments, the therapeutic agent cargos may be retinoids (e.g., retinol, adapalene, tretinoin, isotretinoin), or alpha hydroxy acids. In some embodiments, the modified sophorolipids are preferably tri-substituted with salicylic acid (e.g., compound no. 2). In other embodiments, the modified sophorolipids are preferably di-substituted at R₂ and R₃ (e.g., compound nos. 1,21).

In some embodiments, the modified sophorolipids alone may be topically applied to the affected area of the skin or is prepared into a topical skin care composition comprising additional ingredients. As discussed above, the compositions comprising the modified sophorolipids may be provided in a variety of forms including, but are not limited to, creams, lotions, masks, powders, and cleansers. The compositions according to the invention may be used in a method to remove makeup and other impurities from the face and skin.

In further embodiments, the present invention is directed to a method of treating or preventing a variety of other skin conditions, including hyperpigmentation, wrinkles, and skin looseness. The method comprises topically administering a modified sophorolipid of formula (I) comprising salicylic acid, succinic acid, amino acids, retinol and/or alpha hydroxy acids.

### Method of Maintaining or Restoring Gut Health

In other embodiments, the present invention is directed to a method of maintaining or restoring gut health in a subject. “Maintaining or restoring gut health” means reducing and/or regulating the inflammatory response in the gut and more specifically the epithelial cells, as well as establishing or reestablishing the predominance of beneficial microorganisms within the gut microbial community, or causing the gut microbiome to become healthy and/or balanced.

The method of maintaining or restoring gut health comprises administering to a subject an effective amount of the modified sophorolipid of formula (I) as described herein, having at least one of amino acid, butyric acid, β-hydroxybutyric acid, and phosphoric acid as a therapeutic agent cargo, such as compound Nos. 6-18. In some embodiments, the modified sophorolipids are preferably di-substituted at R₂ and R₃ with β-hydroxybutyric acid (compound no. 10). In other embodiments, the modified sophorolipids are preferably di-substituted at R₂ and R₃ with butyric acid (compound no. 6). In some embodiments, preferred amino acids are glutamine, glutamic acid, arginine, glycine, lysine, threonine, and sulfur-containing amino acids. According to the embodiment, the modified sophorolipid alone may be administered to a subject or is prepared into a suitable composition comprising additional ingredients. The route of administration may be oral or rectal, as long it can efficiently and effectively deliver the therapeutic agent cargo(s) to the subject.

As used herein, reference to the “microbiome,” “microbiota,” “microbial community,” “microflora” or “flora” of the “gut” or of the “GI tract” means the population of microorganisms living within a subject's intestines and/or GI tract. In some embodiments, these microorganisms can also be present in the appendix. A healthy, or balanced, gut microbiome is one that comprises a variety of microbial species, with a majority of those species preferably being beneficial to the health of the subject.

As used herein, a “beneficial” microbe is one that is considered mutualistic, or conferring a benefit to its host, rather than one that is merely commensal (existing within the gut in a non-harmful and non-mutualistic coexistence) or one that is harmful and/or parasitic to the host. Benefits can include, for example, digestion of dietary fiber into short-chain fatty acids and synthesis of certain vitamins.

As used herein, a “disrupted” or “unbalanced” gut microbiome is in dysbiosis, where the species of microbes in a subject's gut comprise an amount, percentage or number of non-beneficial microorganisms that result in disease, discomfort, malnutrition, impaired nutrient absorption, and other deleterious health consequences, that is greater than the amount, percentage, or number of beneficial microorganisms. Non-beneficial microorganisms include harmful microorganisms, such as pathogens and parasites, as well as commensal organisms that do not directly harm the host, but when overgrown, outcompete beneficial gut microorganisms.

Intestinal bacteria are significant components of mammalian metabolic and digestive processes, as well as the health of various other physiological processes. For example, these microbes aide in enterohepatic circulation, and help with the digestion and assimilation of energy sources and essential trace elements into the GI system. Other evidence suggests that gut bacteria may influence inflammation levels, which may affect metabolism and energy balance. A balanced gut microbiome regulates many functions of the immune system, and various other health concerns, ranging from autoimmune diseases to clinical depression and obesity, are be related to immune dysfunction that is often linked to an unbalanced gut microbiome. Additionally, an imbalance of the GI microbiome can compromise the cardiovascular system and has been implicated in the etiology of colorectal cancer and diabetes. Disruptions in the gut microbiome contribute to hormonal disorders, an increase in the appearance of plaques, and the progression of atherosclerosis. There is also evidence for an association between altered gut microbiota and neurodevelopmental (e.g., autism) and neurodegenerative (e.g., Alzheimer's) diseases. Therefore, maintaining or restoring gut health in a subject contributes to the overall health of the subject.

In some embodiments, the methods utilize and enhance the indigenous microorganisms that are present in a subject's GI tract. In certain embodiments, the subject is an animal, preferably a human, whose gut microbiome has been disrupted or unbalanced.

In certain embodiments, the method further comprises administering prebiotics to the subject before, concurrently with, and/or after the administration of the modified sophorolipid to provide an enabling environment for the beneficial microorganisms to grow, and to decrease the amount of time required to restore the gut microbiome. Prebiotics can include, for example, fermentable fibers derived from fructans and xylans, inulin, fructooligosaccharides, xylooligosaccharides and galactooligosaccharides. Foods known to contain prebiotics include, for example, chicory root, onions, garlic, leek, oatmeal, wheat bran, asparagus, dandelion greens, Jerusalem artichoke, and banana.

In one embodiment, the method further comprises introducing a microbial growth by-product that can further enhance the restorative capabilities of the methods. The growth by-products can include those that are produced by the microbes of the high concentration microbial culture, or they can be applied in addition to those produced by the microorganisms. The growth by-products can be biosurfactants, enzymes, biopolymers, solvents, acids, proteins, amino acids, or other metabolites that can be useful for, for example, controlling undesirable microorganisms. In specific embodiments, the growth by-product is a biosurfactant selected from glycolipids (e.g., sophorolipids, rhamnolipids, trehalose lipids and mannosylerythritol lipids) and lipopeptides (e.g., surfactin, iturin, fengycin and lichenysin).

In certain embodiments, the present invention provides a method of reducing pathogenic gram-negative bacteria such as Escherichia coli, Salmonella spp., and Campylobacter spp. in a subject comprising administering to the subject an effective amount of the modified sophorolipid of formula (I) as described herein. Overpopulation of gram-negative bacteria is thought to trigger various ailments including inflammation by promoting synthesis of proinflammatory cytokines. Other examples of pathogenic gram-negative bacteria include Helicobacter pylori, established on gastric mucosa causing chronic gastritis and peptic ulcer disease, and is also a carcinogen for gastric cancer.

As the method according to the present invention maintains and/or restores a healthy microbial balance in the subject's gut, beneficial microorganisms grow within the subject's GI tract following the administration of the modified sophorolipid. The high numbers of the beneficial microorganisms outcompete and/or control the undesirable microorganisms, thus restoring the gut microbiome to a healthy, balanced state.

In one embodiment, the present invention can be used to treat and/or ameliorate the symptoms of health conditions that are a cause and/or a result of a disrupted or unbalanced gut microbiome, such as, for example, Irritable Bowel Syndrome, Type 1 diabetes, Celiac disease, other autoimmune disorders, colorectal cancer, and neurodevelopmental and neurodegenerative diseases, such as Autism Spectrum Disorders and Alzheimer's disease.

In one embodiment, the present invention can be used to treat and/or ameliorate digestive conditions and/or symptoms such as, for example, nausea, vomiting, diarrhea, constipation, gas, bloating, food sensitivities, heartburn, acid-reflux, gastroesophageal reflux disease, indigestion, and abdominal cramps/pain. In one embodiment, the present invention can be used to treat and/or ameliorate extra-intestinal symptoms associated with a variety of health conditions, including symptoms such as, for example, headaches, dizziness, fatigue, backaches, insomnia, eating disorders, nutrient deficiencies, depression, anxiety, fertility issues, joint or muscle pain, brain fog, genital yeast infections, bacterial vaginosis, bladder or urinary tract infections, and many others.

In other embodiments, the present invention can be used to enhance a subject's overall health and well-being. In one embodiment, the present invention can be used to reduce the severity of senescence—or aging-related conditions, wherein the subject is a middle-aged or elderly person, e.g., 50 years of age or older. Aging-related conditions can include, for example, gut dysbiosis, hormonal disruptions, immune decline, stress, infections, bone loss, injuries, organ malfunction, memory loss and many others.

In one embodiment, the present invention can be used to enhance the functioning of a body system, tissue or organ, such as metabolic functions, the digestive system, the immune system, the endocrine system, and the cardiovascular system.

### Method of Improving Weight Gain

Another aspect of the invention is directed to a method of improving weight gain of a subject, the subject preferably being live stocks such as horses, cattle. pigs, sheep, goats, and chickens, and other animals such as mice, rats, guinea pigs, and hamsters. The method comprises administering to a subject an effective amount of the modified sophorolipid of formula (I) having at least one of amino acid, butyric acid, β-hydroxybutyric acid, phosphoric acid as a therapeutic agent cargo, such as compound Nos. 6-18. In some embodiments, the modified sophorolipids are preferably di-substituted at R₂ and R₃ with butyric acid (compound no. 6). According to the embodiment, the modified sophorolipid alone may be administered to a subject or is prepared into a suitable composition comprising additional ingredients. The route of administration may be oral or rectal, as long it can efficiently and effectively deliver the therapeutic agent cargo(s) to the subject.

Without being bound by theory, the method according to the invention improves the subject's weight gain by increasing feed intake and conversion as well as the production of growth hormone (e.g., GH/IGF-1) in the subject. Additionally, weight gain is also enhanced through an improvement in the subject's gut flora, reduction in the gram-negative bacteria, and control of inflammation.

Growth as used herein can be defined as an increase in live weight gain per unit of time. Growth can also be defined as a progressive increase in size (length, height, girth, volume) or weight of an animal during a specific time period.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention, e.g., the ability to improve the bioavailability of a substance. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. As further examples, “about” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Examples

Following are exemplary compounds of the present invention which are offered by way of illustration and are not intended to limit the invention. The present invention covers all of the compounds represented by the general formula (I), including salts thereof, hydrates thereof, geometric and optical isomers thereof, and polymorphic forms thereof. Various modifications or changes within the spirit and purview of this application will be suggested to and understood by persons skilled in the art.

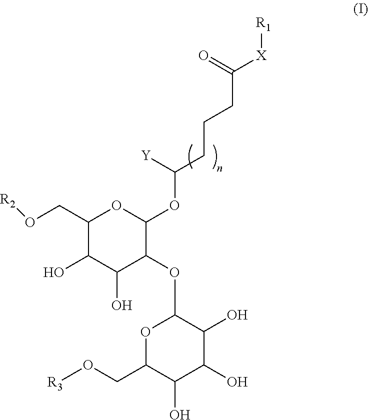

Y is methyl.
n=12; unsaturation=0-3.

The numbers in the left most column represent compound numbers.

| No. | R₁ | R₂ | R₃ | X |
|---|---|---|---|---|
| 1 | H | 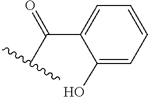 [SMILES: *C(=O)c1ccccc1O] | 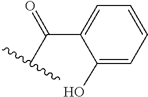 [SMILES: *C(=O)c1ccccc1O] | O |
| | | Salicylic acid cargo | Salicylic acid cargo | |
| 2 | 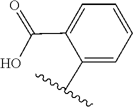 [SMILES: *c1ccccc1C(=O)O] | 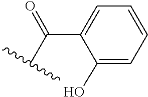 [SMILES: *C(=O)c1ccccc1O] | 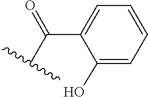 [SMILES: *C(=O)c1ccccc1O] | O |
| | Salicylic acid cargo | Salicylic acid cargo | Salicylic acid cargo | |
| 3 | 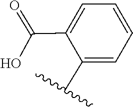 [SMILES: *c1ccccc1C(=O)O] | H or acetyl | H or acetyl | O |
| | Salicylic acid cargo | | | |
| 4 | H | 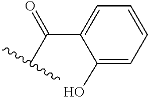 [SMILES: *C(=O)c1ccccc1O] | H or acetyl | O |
| | | Salicylic acid cargo | | |
| 5 | H | H or acetyl | 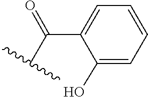 [SMILES: *C(=O)c1ccccc1O] | O |
| | | | Salicylic acid cargo | |
| 6 | H | 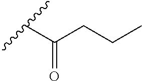 [SMILES: *C(=O)CCC] | 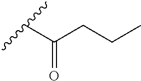 [SMILES: *C(=O)CCC] | O |
| | | Butyric acid cargo | Butyric acid cargo | |
| 7 | 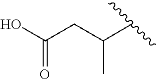 [SMILES: *C(C)CC(=O)O] | 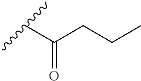 [SMILES: *C(=O)CCC] | 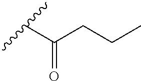 [SMILES: *C(=O)CCC] | O |
| | β-hydroxybutyric acid | Butyric acid cargo | Butyric acid cargo | |
| | cargo | | | |
| 8 | H | H or acetyl | 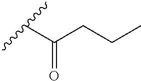 [SMILES: *C(=O)CCC] | O |
| | | | Butyric acid cargo | |
| 9 | 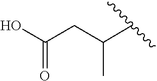 [SMILES: *C(C)CC(=O)O] | H or acetyl | H or acetyl | O |
| | β-hydroxybutyric acid | | | |
| | cargo | | | |
| 10 | H | 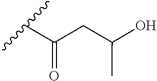 [SMILES: *C(=O)CC(C)O] | 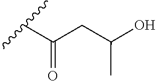 [SMILES: *C(=O)CC(C)O] | O |
| | | β-hydroxybutyric acid cargo | β-hydroxybutyric acid | |
| | | | cargo | |
| 11 | H | H or acetyl | 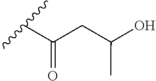 [SMILES: *C(=O)CC(C)O] | O |
| | | | β-hydroxybutyric acid | |
| | | | cargo | |
| 12 | H | 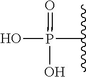 [SMILES: *P(=O)(O)O] | 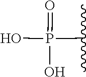 [SMILES: *P(=O)(O)O] | O |
| | | Phosphoric acid cargo | Phosphoric acid cargo | |
| 13 | H | 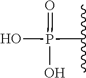 [SMILES: *P(=O)(O)O] | H or acetyl | O |
| | | Phosphoric acid cargo | | |
| 14 | H | H or acetyl | 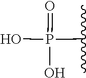 [SMILES: *P(=O)(O)O] | O |
| | | | Phosphoric acid cargo | |
| 15 | H | 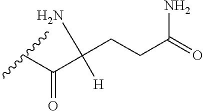 [SMILES: *C(=O)C(N)CCC(N)=O] | 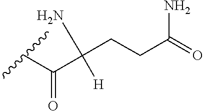 [SMILES: *C(=O)C(N)CCC(N)=O] | O |
| | | Glutamine cargo | Glutamine cargo | |
| 16 | 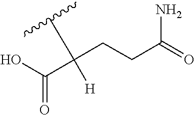 [SMILES: *C(CCC(N)=O)C(=O)O] | 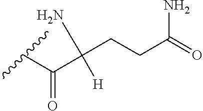 [SMILES: *C(=O)C(N)CCC(N)=O] | 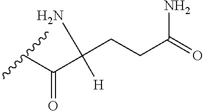 [SMILES: *C(=O)C(N)CCC(N)=O] | NH |
| | Glutamine cargo | Glutamine cargo | Glutamine cargo | |
| 17 | H | 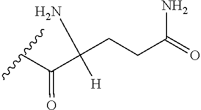 [SMILES: *C(=O)C(N)CCC(N)=O] | H or acetyl | 0 |
| | | Glutamine cargo | | |
| 18 | 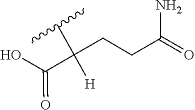 [SMILES: *C(CCC(N)=O)C(=O)O] | H or acetyl | H or acetyl | NH |
| | Glutamine cargo | | | |
| 19 | H | 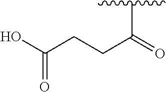 [SMILES: *C(=O)CCC(=O)O] | H or acetyl | O |
| | | Succinic acid cargo | | |
| 20 | H | H or acetyl | 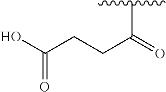 [SMILES: *C(=O)CCC(=O)O] | O |
| | | | Succinic acid cargo | |
| 21 | H | 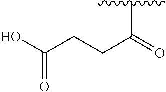 [SMILES: *C(=O)CCC(=O)O] | 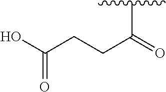 [SMILES: *C(=O)CCC(=O)O] | O |
| | | Succinic acid cargo | Succinic acid cargo | |
| 22 | 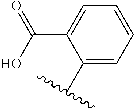 [SMILES: *c1ccccc1C(=O)O] | 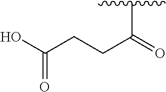 [SMILES: *C(=O)CCC(=O)O] | 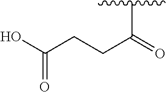 [SMILES: *C(=O)CCC(=O)O] | O |
| | Salicylic acid cargo | Succinic acid cargo | Succinic acid cargo | |
| 23 | 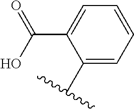 [SMILES: *c1ccccc1C(=O)O] | 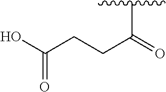 [SMILES: *C(=O)CCC(=O)O] | H or acetyl | O |
| | Salicylic acid cargo | Succinic acid cargo | | |
| 24 | 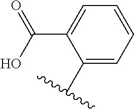 [SMILES: *c1ccccc1C(=O)O] | H or acetyl | 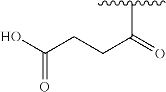 [SMILES: *C(=O)CCC(=O)O] | O |
| | Salicylic acid cargo | | Succinic acid cargo | |

1. A compound of formula (I) or a salt thereof: 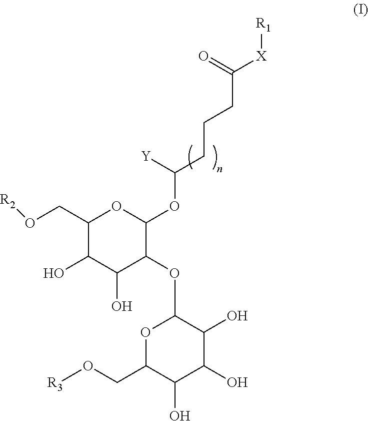
wherein:
Y is hydrogen or methyl;
R₁ is a first therapeutic agent cargo linked via an ester linkage with X═O or an amide linkage with X═NR₀, wherein R₀ is hydrogen or a substituent; or R₁ is hydrogen and X is O;
R₂ and R₃ are each independently hydrogen, acetyl, or a second therapeutic agent cargo linked via an ester linkage; and
n is an integer from 4 to 16, and the chain defined by n has 0-3 unsaturation,
wherein at least one of R₁, R₂, and R₃ is a therapeutic agent cargo.

2. The compound according to claim 1, wherein Y is methyl, n is 12, and the chain defined by n has at least 1 unsaturation that is a double bond.

3. The compound according to claim 1, wherein:
the first therapeutic agent cargo is salicylic acid, β-hydroxybutyric acid, amino acid, or succinic acid; and
the second therapeutic agent cargo is salicylic acid, butyric acid, β-hydroxybutyric acid, amino acid, succinic acid, or phosphoric acid,
wherein the first and second therapeutic agent cargos may be same or different.

4. The compound according to claim 1, as a salt wherein X is O⁻ and R₁ is lithium (Li⁺), sodium (Na⁺), or potassium (K⁺).

5. The compound according to claim 1, wherein the efficacy of the first and/or second therapeutic agent cargo is enhanced.

6. The compound according to claim 1, wherein:
R₁ is a first therapeutic agent cargo linked via an ester linkage with X═O or an amide linkage with X═NR₀, wherein R₀ is hydrogen, lower alkyl, aryl, or a combination thereof; and
R₂ and R₃ are each independently hydrogen or acetyl.

7. The compound according to claim 6, wherein:
the first therapeutic agent cargo is salicylic acid, β-hydroxybutyric acid, amino acid, or succinic acid.

8. The compound according to claim 6, wherein the efficacy of the first therapeutic agent cargos is enhanced.

9. The compound according to claim 1, wherein:
R₁ is a first therapeutic agent cargo linked via an ester linkage with X═O or an amide linkage with X═NR₀, wherein R₀ is hydrogen, lower alkyl, aryl, or a combination thereof;
R₂ and R₃ are each independently hydrogen, acetyl, or a second therapeutic agent cargo linked to the sophorolipid structure via an ester linkage, provided that at least one of R₂ and R₃ is the second therapeutic agent cargo; and
wherein the first and second therapeutic agent cargos may be same or different.

10. The compound according to claim 9, wherein:
the first therapeutic agent cargo is salicylic acid, β-hydroxybutyric acid, amino acid, or succinic acid; and
the second therapeutic agent cargo is salicylic acid, butyric acid, β-hydroxybutyric acid, amino acid, succinic acid, or phosphoric acid,
wherein the first and second therapeutic agent cargos may be same or different.

11. The compound according to claim 9, wherein the efficacy of the first and/or second therapeutic agent cargos is enhanced.

12. The compound according to claim 1, wherein:
R₁ is hydrogen and X is O; and
R₂ and R₃ are each independently hydrogen, acetyl, or a second therapeutic agent cargo linked to the sophorolipid structure-via an ester linkage, provided that at least one of R₂ and R₃ is the second therapeutic agent cargo.

13. The compound according to claim 12, wherein:
the second therapeutic agent cargo is salicylic acid, butyric acid, β-hydroxybutyric acid, amino acid, succinic acid, or phosphoric acid.

14. The compound according to claim 12, wherein the efficacy of the second therapeutic agent cargos is enhanced.

15. A method of improving the bioavailability and/or dermal penetrability of an active ingredient in a subject comprising administering to the subject an effective amount of the compound according to claim 1, wherein said active ingredient is the first therapeutic agent cargo and/or the second therapeutic agent cargo.

16. A method of treating a skin condition or ailment of a subject by administering to the subject an effective amount of the compound according to claim 1, wherein the first therapeutic agent cargo and the second therapeutic agent cargo are each independently salicylic acid or succinic acid.

17. The method according to claim 16, wherein the skin condition or ailment is acne.

18. A method of maintaining or restoring gut health in a subject comprising administering to the subject an effective amount of the compound according to claim 1, wherein the first therapeutic agent cargo and the second therapeutic agent cargo are each independently amino acid, butyric acid, β-hydroxybutyric acid, or phosphoric acid.

19. A method of reducing pathogenic gram-negative bacteria in the microbiome of a subject comprising administering to the subject an effective amount of the compound according to claim 1, wherein the first therapeutic agent cargo and the second therapeutic agent cargo are each independently amino acid, butyric acid, β-hydroxybutyric acid, or phosphoric acid.

20. A method of improving weight gain of a subject comprising administering to the subject an effective amount of the compound according to claim 1, wherein the first therapeutic agent cargo and the second therapeutic agent cargo are each independently amino acid, butyric acid, β-hydroxybutyric acid, or phosphoric acid.
